(19) United States
(12) Patent Application Publication
(10) Pub. No.: US 2014/0011761 A1
(43) Pub. Date: Jan. 9, 2014

(54) Modulating Endoplasmic Reticulum Stress in the Treatment of Tuberous Sclerosis

(71) Applicant: President and Fellows of Harvard College, Cambridge, MA (US)

(72) Inventors: Gokhan S. Hotamisligil, Wellesley, MA (US); Umut Ozcan, Jamaica Plain, MA (US); Brendan D. Manning, Boston, MA (US)

(73) Assignee: President and Fellows of Harvard College, Cambridge, MA (US)

(21) Appl. No.: 13/722,180

(22) Filed: Dec. 20, 2012

Related U.S. Application Data
(63) Continuation of application No. 12/092,345, filed on Nov. 21, 2008, now abandoned.
(60) Provisional application No. 60/732,334, filed on Nov. 1, 2005.

(51) Int. Cl. A61K 31/343; A61K 45/06; G01N 33/68; A61K 31/706; A61K 31/192

(52) U.S. Cl. CPC A61K 31/343; A61K 31/706; A61K 31/192; G01N 33/6893; A61K 45/06
USPC 514/35; 514/468; 514/570; 514/182; 514/645; 435/7.4; 435/7.92; 514/171

(57) ABSTRACT

Endoplasmic reticulum stress has been found to be associated with the genetic disease tuberous sclerosis. Tuberous sclerosis is cause by defects in the two genes, TSC1 and TSC2. Agents that modulate ER stress may be used to treat tuberous sclerosis and other hamartomatous diseases. In particular, 4-phenyl butyric acid (PBA) has been shown to reduce ER stress is TSC-deficient cells. Other compounds useful in reducing ER stress are chemical chaperones such as trimethylamine N-oxide arid glycerol may also be useful in treating tuberous sclerosis. The present invention provides methods of treating a subject suffering from tuberous sclerosis using ER stress reducers such as PBA, TUDCA, UDCA, and TMAO. Methods of screening for ER stress reducers by identifying agents that reduce levels of ER stress markers in TSC-deficient cells are also provided. These agents may find use in methods and pharmaceutical compositions for treating tuberous sclerosis.

### RELATED APPLICATIONS

The present claims priority to and the benefit of U.S. provisional patent application Ser. No. 60/732,334, filed Nov. 1, 2005, the entire contents of which are incorporated herein by reference.

### FIELD OF THE INVENTION

The invention generally relates to a method for the prevention, alleviation and/or treatment of hamartomatous diseases using compounds that modulate endoplasmic reticulum (ER) stress. More specifically, the invention relates to the prevention, alleviation, and/or treatment of tuberous sclerosis using chemical chaperones or agents that promote ER stress. The invention further relates to methods for screening compounds that modulate ER stress using cells containing a mutation in a tuberous sclerosis complex (TSC) gene.

### BACKGROUND

Tuberous sclerosis, also called tuberous sclerosis complex (TSC), is a rare, inherited hamartomatous disease associated with multiple tumors and neurological disorders. The tumors may be found in the brains (e.g., cortical tubers, subependymal nodules, and giant-cell astrocytomas) and on other vital organs such as the kidneys (e.g., angiomyolipomas), heart (e.g., cardiac rhabdomyomas), eyes (e.g., phakomas), lungs, liver, pancreas, and skin (e.g., hypomelanic macules, facial angiofibromas, forehead plaques, shagreen patches, ungual and subungual fibromas, molluscum fibrosum, café au lait spots, and poliosis). The tumors in tuberous sclerosis are rarely malignant however. The disease commonly affects the central nervous system leading to neurological problems such as seizures, mental retardation, and behavior problems. Bone cysts, rectal polyps, gum fibromas, and dental pits are also seen in patients with tuberous sclerosis (Kwiatkowski, “Tuberous sclerosis: from Tubers to mTOR” Annals of Human Genetics 67:87-96, 2003; Lendvay et al. “The Tuberous Sclerosis Complex and Its Highly Variable Manifestations” J. Urology 169:1635-42, 2003; each of which is incorporated herein by reference).

Although tuberous sclerosis may be present at birth, the signs of the disorder may be subtle and full symptoms may take some time to develop. As a result, tuberous sclerosis is frequently unrecognized and/or misdiagnosed for years.

Tuberous sclerosis affects an estimated 25,000 to 40,000 individuals in the United States and about 1 to 2 million individuals worldwide. Tuberous sclerosis is seen in approximately 1 out of 6,000 newborns. The disorder has been found in all races and ethnic groups, and in both genders. No racial or sex predilections have been observed. The disorder was once known as epiloia or Bourneville's disease.

Most cases of tuberous sclerosis occur as spontaneous mutations. However, the disease may be inherited from a parent with tuberous sclerosis. Individuals who inherit tuberous sclerosis may not have the same symptoms as their parent with the disorder.

The genes causing tuberous sclerosis have been identified and named TSC1 and TSC2 (van Slegtenhorst et al. “Identification of the tuberous sclerosis gene TSC1 on chromosome 9q34” Science 277:805-8, Aug. 8, 1997; European Chromosome Tuberous Sclerosis Consortium, “Identification and Characterization of the Tuberous Sclerosis Gene on Chromosome 16” Cell 75:1305-1315, 1993; each of which is incorporated herein by reference). Only one of the genes needs to be affected for tuberous sclerosis to be present. The TSC1 gene was discovered in 1991 and is found on chromosome 9, specifically 9q34. It produces a protein call hamartin. The second gene, TSC2, was discovered in 1993. It is found on chromosome 16, specifically 16p13, and produces the protein tuberin.

Although treatments are available for a number of the symptoms of tuberous sclerosis, there is no treatment or cure for the underlying causes of the disease. For example, anti-epileptic drugs may be used to control seizures, and surgery may be useful for treating the tumors and skin lesions. There remains a need for improved treatments of tuberous sclerosis.

### SUMMARY OF THE INVENTION

The invention includes a method for the prevention, alleviation, and/or treatment of hamartomatous disease, particularly tuberous sclerosis, using agents to decrease ER stress such as chemical chaperones. The present invention stems from the recognition that tuberous sclerosis is associated with endoplasmic reticulum (ER) stress. Mutations in TSC1 and TSC2, the genes identified as causing tuberous sclerosis, result in uncontrolled activity of the mammalian target of rapamycin (mTOR) signaling pathway that results in severe ER stress. Tumors derived from a mouse model of tuberous sclerosis were found to exhibit high levels of ER stress. Furthermore, ER stress was also evident in human tumors of patients with tuberous sclerosis. Endoplasmic reticulum stress associated with tuberous sclerosis was reversed by the treatment of cells with agents that reduce ER stress (e.g., chemical chaperones), thereby providing a new treatment option for tuberous sclerosis patients.

The invention further includes a method for the prevention, alleviation, and/or treatment of hamartomatous disease, particularly tuberous sclerosis by administration of agents to induce ER stress. It has been found that the TSC-deficient cells are highly sensitive to ER stress and can be selectively killed by treatment with low doses of ER-stress inducing agents that do not affect normal tissues at comparable doses. These results have been confirmed in animal studies in which administration of a low dose of an ER stress inducing agent was demonstrated to increase cell death in kidney and liver tumors in TSC-deficient mice. No increase in apoptosis was observed in normal tissue in response to the agent.

The invention also includes a method for promoting apoptosis in TSC-deficient cells comprising administration of a dose of an ER-stress inducing agent at a level which the ER stress inducing agent does not induce significant apoptosis in normal (e.g., wild-type) or non-tumor cells or tissues. Tumors associated with tuberous sclerosis can be treated by administering to a subject an agent that increases the ER stress response (e.g., thapsigargin, tunicamycin, azetidine-2 carboxylic acid (Azc, a purine analog)) to eliminate TSC-deficient cells.

In another aspect of the present invention, TSC1- and TSC2-deficient cells are used as a cell model of spontaneous ER stress. These cells can be used in a system to investigate ER stress in cells or to identify new ER stress modulators. For examples, these cells can be used in screening methods, including high throughput screening methods, to identify agents that modulate the ER stress response. In a screening method of the invention, test compounds are contacted, preferably independently, with TSC1- and/or TSC2-deficient cells, and analyzed for a change in the levels of at least one ER stress marker as compared to control cells or samples. ER stress markers include spliced forms of Xbox binding protein-1 (XBP-1); phosphorylated protein kinase-like ER kinase (PERK), eukaryotic initiation factor 2α (eIF2α), and inositol requiring enzyme (IRE-1); increased mRNA and/or protein expression of GRP78/BIP and C/EPB homologous protein (CHOP); and c-Jun N-terminal kinase (JNK) activation. Analysis of the level of at least one marker in response to a test compound is used to identify agents that modulate (i.e., increase or decrease) ER stress. The identified agents are also part of the present invention. The test compounds include, but are not limited to, small molecules, polynucleotides, proteins, and peptides. In certain embodiments, the test compounds are small molecules. In other embodiments, the test compounds are polynucleotides such as siRNAs.

The identified agents may be used in pharmaceutical compositions to treat any disease or condition associated with ER stress (e.g., tuberous sclerosis, hypercholesterolemia, hyperlipidemia, type II diabetes, obesity, etc.) ER stress results from a disequilibrium between ER load and folding capacity, and can be triggered by any of a number of factors including hypoxia, hypoglycemia, toxins, and genetic predisposition. In certain embodiments, the agents are administered to a subject in therapeutically effective amounts to prevent, alleviate, and/or treat a disease or condition associated with ER stress.

Agents useful in the treatment of tuberous sclerosis include small molecules, proteins, nucleic acids, and any other chemical compounds known to reduce or prevent ER stress. These agents may act in any manner that reduces or prevents ER stress such as reducing the production of mutant or misfolded proteins, increasing the expression of ER chaperones, increasing the stability of proteins, boosting the processing capacity of the ER, etc. Particularly useful agents include chemical chaperones such as 4-phenyl butyrate (PBA), tauroursodeoxycholic acid (TUDCA), ursodeoxycholic acid (UDCA), trimethylamine oxide (TMAO), glycerol, D₂O, dimethylsufloxide, glycine betaine, methyl amines, and glycerophosphocholine. The invention includes the use of such chemical chaperones for the preparation of medicament for the treatment of diseases associated with ER stress, particularly tuberous sclerosis and other hamartomatous diseases. In particular, both PBA and TUDCA have been shown to regulate ER stress in animals as measured by the reduced phosphorylation of PERK, reduced activation of JNK, and reduced phosphorylation of IRE-1α, as determined by western blot after treatment of the animal with the compound. The agent or a pharmaceutical composition comprising the agent is administered to a subject (e.g., human, dog, cat, mammal, animal) in doses effective to reduce ER stress, and thereby treat tuberous sclerosis and other hamartomatous disease.

The invention also provides methods of alleviating, treating, and/or preventing tuberous sclerosis by administering agents that reduce ER stress. The agents may be administered in any manner known in the drug delivery art although preferably the agent is delivered orally or parenterally. Dose ranges for these agents depend on the agent being delivered as well as other factors but will typically range from about 1 mg/kg/day to about 10 g/kg/day, but may be dosed at other levels. These agents, pharmaceutical compositions, and treatment methods may also be used in the treatment of other hamartomatous diseases such as pulmonary hamartoma, von Meyenburg complex, proteus syndrome, Birt-Hogg-Dube syndrome, multiple hamartoma syndrome, neurofibromatosis type 1, Peutz-Jeghers syndrome, Riley-Smith syndrome, and angiomyolipoma.

The effectiveness of the treatment using ER stress modulators may be monitored by determining the levels of at least one ER stress marker in the subject being treated. In certain embodiments, reduced indicators of ER stress indicate the treatment is working. In other embodiments, such as inducing apoptosis in tuberous sclerosis tumors, increased indicators of ER stress, cell death, or apoptosis indicate that the treatment is working. Such distinctions can be readily made by those skilled in the art.

In certain embodiments, the agent used to treat tuberous sclerosis is 4-phenyl butyric acid (PBA).

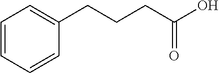 [SMILES: O=C(O)CCCc1ccccc1]

PBA has been shown to regulate ER stress. Phenyl butyric acid (PBA), or a derivative or salt thereof, is administered to a subject in order to reduce ER stress and is particularly useful in the treatment of tuberous sclerosis. The administration of PBA results in a reduction in the signs and symptoms of tuberous sclerosis. In certain embodiments, PBA prevents or slows the growth of tumors associated with tuberous sclerosis. Increased ER stress in TSC2−/− cells is inhibited by treatment with PBA. PBA, or a pharmaceutical composition thereof, is administered in doses ranging from about 10 mg/kg/day to about 2 g/kg/day, preferably from about 100 mg/kg/day to about 1 g/kg/day, more preferably from about 500 mg/kg/day to about 1 g/kg/day.

In another embodiment, tauroursodeoxycholic acid (TUDCA), a bile acid, is the agent used to treat tuberous sclerosis.

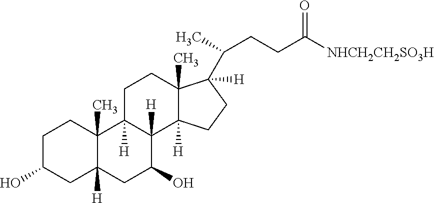

TUDCA has been shown to regulate ER stress. The invention provides the administration of tauroursodeoxycholic acid (TUDCA) or a salt or derivative thereof to a subject in order to reduce ER stress. TUDCA, or a pharmaceutical composition thereof, is administered in doses ranging from about 10 mg/kg/day to about 2 g/kg/day, preferably from about 100 mg/kg/day to about 1 g/kg/day, more preferably from about 250 mg/kg/day to about 750 mg/kg/day.

In another embodiment, TMAO is the agent used to reduce ER stress. The invention provides the administration of TMAO or a salt or derivative thereof to a subject in order to reduce ER stress. TMAO, or a pharmaceutical compositions thereof, is administered in doses ranging from 10 mg/kg/day to 5 g/kg/day, preferably from 100 mg/kg/day to 1 g/kg/day, more preferably from 250 mg/kg/day to 750 mg/kg/day.

Pharmaceutical compositions and medicaments including agents that modulate ER stress (e.g., PBA, TUDCA, UDCA, TMAO, thapsigargin, tunicamycin, azetidine-2 carboxylic acid (Azc)) and pharmaceutically acceptable excipients are also provided. The pharmaceutical compositions may be formulated for oral, parenteral, or transdermal delivery. The ER stress reducing agent may also be combined with other pharmaceutical agents. The agents may be combined in the same pharmaceutical composition or may be kept separate (i.e., in two separate formulations) and provided together in a kit. The kit may also include instructions for the physician and/or patient, syringes, needles, box, bottles, vials, etc.

In another aspect, the invention provides a method of screening for agents that reduce ER stress and that are useful in the treatment of tuberous sclerosis or other hamartomatous diseases. Agents to be screened are contacted with at least one TSC-deficient cell and preferably at least one control cell. In certain embodiments, TSC2−/− cells are used in screening for agents that modulate ER stress. In another embodiment, TSC1−/− cells are used in screening for agents that modulate ER stress. In yet another embodiment, heterozygous TSC-deficient cells are used in screening for agents that modulate ER stress. Cells may be primary cells obtained from an animal or patient, or tissue culture cells modified to include mutations and/or deletions in at least one of the TSC genes. Cells particularly useful in the inventive screen include mammalian cells, particularly human cells. The levels of ER stress markers are compared to levels of the same stress marker in control cells or tissues to identify agents that reduce ER stress. The selection and use of control samples is well understood by those skilled in the art. Examples of markers of ER stress include spliced forms of XBP-1, phosphorylated PERK, eIF2α, and IRE-1α; increased mRNA and/or protein levels of GRP78/BIP and CHOP; reduced insulin signaling; and JNK activation. Agents that reduce at least one marker of ER stress as compared to an untreated control cell are identified as agents that reduce ER stress. A decrease in the levels of an ER stress marker is indicative of an agent that can be useful in treating diseases associated with ER stress, such as tuberous sclerosis and other hamartomatous diseases. It is understood that a similar screening method can be used for agents that cause increased ER stress. In such a screening method, cells may be exposed to conditions or compounds that induce ER stress, including, but not limited to, glucose starvation in TSC-deficient cells, or treatment with ER stress inducing agents of normal cells, prior to, simultaneous with, and/or after exposure to test agents. Agents identified using the inventive method are part of the invention. These agents may be further tested for use in pharmaceutical compositions and medicaments. The invention further includes a kit for screening agents to modulate ER stress.

In another aspect, the invention provides a method of diagnosing tuberous sclerosis, monitoring the progression of the disease, or monitoring treatment of the disease by analyzing levels of ER stress markers. Markers indicative of ER stress include, but are not limited to, spliced forms of XBP-1; phosphorylated PERK, eIF2α and IRE-1α; increased mRNA and/or protein levels of GRP78/BIP and/or CHOP; decreased insulin signaling; and JNK activation. In a preferred embodiment, the amount of marker present in a test sample suspected of undergoing ER stress (e.g., tumor tissue from an individual having a TSC-deficiency) is compared to normal tissue, preferably from a site close to or adjacent to the test sample to be analyzed. Any cellular marker known to be indicative of ER stress (e.g., components of the UPR) may also be used to identify ER stress. The levels of these markers may be measured by any method known in the art including western blot, enzyme-linked immunosorbent assay (ELISA), northern blot, immunoassay, immunohistochemistry, rtPCR, especially quantitative rtPCR, PCR in situ, or enzyme assay. The specific method of detection of an altered level of at least one ER stress marker is not a limitation of the invention. In the diagnostic method, an increase in the level of an ER stress markers indicates that the subject is at risk for tuberous sclerosis or other harmatomatous disease. In monitoring the progression of the disease or the treatment, a reduction in ER stress markers indicates a reduction in the progression of the disease or a success in treating the disease with ER stress reducers. When ER stress inducers are used to induce cell death in tuberous sclerosis-associated tumors, an increase in ER stress markers indicates a successful treatment, however a more likely endpoint for monitoring would be an increase in cell death (e.g., apoptosis).

### DEFINITIONS

“Alleviate”: The term alleviate, as used herein, is understood as to make a condition, such as tuberous sclerosis or other hamartomatous disease, more bearable, and/or to partially remove or correct at least one symptom and/or hallmark of the disease. Alleviation of a disease or symptoms thereof does not require curing the disease or completely eliminating any or all of the symptoms of the disease. More than one dose of an agent that modulates ER stress may be required for the alleviation of disease.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). In certain embodiments, the animal is a human.

“Cell”: As used herein, the term “cell” includes any cell. In certain embodiments, the cell is prokaryotic. In other embodiments, the cell is eukaryotic. In one embodiment, a cell of the invention is a bacterial cell. In another embodiment, a cell of the invention is a fungal cell, such as a yeast cell. In another embodiment, a cell of the invention is a vertebrate cell, e.g., an avian or mammalian cell. In yet another embodiment, a cell of the invention is a human cell. In certain embodiments, the cell is derived for a tumor (e.g., angiomyolipomas, brain tumors, cortical tumors, subependymal nodules, giant-cell astrocytomas, rhabdomyomas, phakomas, facial angiofibromas, ungual or subungual fibromas, molluscum fibrosum, etc.) associated with tuberous sclerosis. Numerous cell types can be used in the inventive screening system. In certain embodiments, the cells are TSC-deficient (e.g. TSC1−/−, TSC1+/−, TSC2−/−, or TSC2+/−).

“Chemical chaperone”: As used herein, a “chemical chaperone” is one of a chemically diverse class of compounds known to increase ER capacity, stabilize protein conformation against denaturation, and/or to facilitate protein folding or re-folding, thereby preserving and/or maintaining protein structure and function (Welch et al. Cell Stress Chaperones 1:109-115, 1996; incorporated herein by reference). In certain embodiments, the “chemical chaperone” is a small molecule or low molecular weight compound. Preferably, the “chemical chaperone” is not a protein. Examples of “chemical chaperones” include, but are not limited to glycerol, deuterated water (D₂O), dimethylsulfoxide (DMSO), trimethylamine N-oxide (TMAO), glycine betaine (betaine), glycerolphosphocholine (GPC) (Burg et al. Am. J. Physiol. (Renal Physiol. 43):F762-F765, 1998; incorporated herein by reference), 4-phenyl butyrate or 4-phenyl butyric acid (PBA), methylamines, ursodeoxycholic acid (UDCA), and tauroursodeoxycholic acid (TUDCA). Chemical chaperones may be used to influence the protein folding in a cell. Chemical chaperones have been shown in certain instances to correct folding/trafficking defects seen in such diseases as cystic fibrosis (Fischer et al. Am. J. Physiol. Lung Cell Mol. Physiol. 281:L52-L57, 2001; incorporated herein by reference), prion-associated diseases, nephrogenic diabetes insipidus, and cancer (Bal et al. Journal of Pharmacological and Toxicological Methods 40:39-45, July 1998; incorporated herein by reference). Chemical chaperones also find use in the reduction of ER stress and are useful in the treatment of obesity, type II diabetes, insulin resistance, and hyperglycemia (See, e.g., Özcan et al., Science 313:1137-40, 2006; PCT/US2005/032840 and PCT/US2005/032841, all three of which are incorporated herein by reference). Preferred chemical chaperones of the instant invention include compounds that decrease the level of ER stress as determined by a decrease in the level of at least one ER stress marker in cells as compared to the level of the marker in cells prior to exposure to the chemical chaperone. ER stress can be due to stress (e.g., hypoxia, hypoglycemia), chemical stimulation, or the presence of a mutation in the cell that results in ER stress.

“Effective amount”: In general, the “effective amount” of an active agent, such as an ER stress reducer or a pharmaceutical composition thereof, refers to the amount of the active agent necessary to elicit the desired biological response. As will be appreciated by those of ordinary skill in this art, the effective amount of an agent that modulates ER stress may vary depending on such factors as the desired biological endpoint, the agent being delivered, the disease being treated, the subject being treated, route of administration etc. An effective amount is not a specific dose or dosage regimen, but instead it is the amount determined by a qualified individual, such as a physician, to be an appropriate dose for an individual for the prevention, alleviation, and/or treatment of a disease associated with ER stress, particularly tuberous sclerosis. For example, the effective amount of agent used to treat tuberous sclerosis is the amount that results in a reduction in the signs and symptoms of the disease (e.g., tumor growth, number of tumors, severity of seizures, number of seizures, progression of renal disease, etc.). In other embodiments, the effective amount of the ER stress modulator reduces the levels of at least one ER stress marker. In certain embodiments, the levels of at least two, three, four, or more ER stress markers are reduced. The ER stress marker may be reduced by approximately 10%, 20%, 30%, 40%, 50%, 60%, 70%, 80%, 90%, 95%, 98%, 99%, or 100%.

“Endoplasmic reticulum (ER) stress inducing agent” as used herein refers to any of a number of chemically diverse compounds that increase the level of stress in the ER as determined by an increase in at least one ER stress marker in cells as compared to the level of the ER stress marker prior to exposure to the ER stress inducing agent. Cells include those already undergoing ER stress. ER stress inducing agents include, but not limited to, thapsigargin, tunicamycin, azetidine-2 carboxylic acid (Azc, a purine analog).

“Endoplasmic reticulum (ER) stress markers” as used herein refers to the hallmarks of ER stress, such as those observed in TSC-deficient cells. Markers can be proteins that are modified (e.g., phosphorylated or dephosphorylated) or translocated in response to ER stress (e.g., translocation of spliced forms of XPB-1 to the nucleus). mRNA and/or protein levels, or mRNA splicing may also be altered in response to ER stress resulting in the production of different amounts or isoforms of proteins. ER stress markers, but are not limited to, an increased amount of spliced XBP-1 as compared to unspliced XBP-1; phosphorylated PERK, eIF2α, and IRE-1α; increased expression of GRP78/BIP and CHOP mRNA and protein; decreased insulin signaling; increased expression of mRNA and protein of components of the UPR; and JNK activation. The level of an ER stress markers in a cell suspected of undergoing ER stress is compared to level of the same ER stress marker in control cells not undergoing ER stress (e.g., cells contacted with a test agent are compared to cells not exposed to a test agent; tumor cells are compared to normal cells, preferably from the same tissue). Each ER stress marker can be assessed individually.

“Endoplasmic reticulum (ER) stress modulating agent” as used herein refers to any of a number of chemically diverse compounds that increase or decrease the level of stress in the ER as determined by a change in the level of at least one ER stress marker in normal cells or cells undergoing ER stress due to mutation and/or chemical stimulation. ER stress modulating agents include chaperones, especially chemical chaperones, that reduce the level of ER stress. ER stress modulating agents include agents that increase ER stress including, but not limited to, thapsigargin, tunicamycin, azetidine-2 carboxylic acid (Azc, a purine analog).

“Peptide” or “protein”: According to the present invention, a “peptide” or “protein” comprises a string of at least three amino acids linked together by peptide bonds. The terms “protein” and “peptide” may be used interchangeably. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide” refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O-(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

“Prevent”: As used herein, the term “prevent” is understood as to keep at least one symptom and/or hallmark of a disease, particularly tuberous sclerosis, from happening or existing in a subject. Prevention can be understood in limiting spreading or exacerbation of symptoms and/or hallmarks of the disease in a subject already diagnosed with tuberous sclerosis or other hamartomatous disease. More than one dose of an agent that modulates ER stress may be required for the prevention of disease.

“Significant apoptosis:” As used herein, the term “significant apoptosis” is understood as less than about 5%, preferably less that about 3%, most preferably less than about 1% of the amount of apoptosis observed in a representative sample TSC-deficient cells or tissues, or other cells undergoing ER stress. ER stress inducing agents are often toxic, and apoptosis occurs naturally in cells and tissues. Therefore, is it possible that ER stress inducing agents can be specific for inducing apoptosis in TSC-deficient cells or cells undergoing ER stress so long as significant apoptosis is not observed in normal control or adjacent, non-tumor cells or tissue.

“Small molecule”: As used herein, the term “small molecule” refers to organic compounds, whether naturally-occurring or artificially created (e.g., via chemical synthesis) that have relatively low molecular weight (e.g., less than about 7500, less than about 5000, less than about 1000 molecular weight or less than about 500 molecular weight) and that are not proteins, polypeptides, or nucleic acids. Typically, small molecules have a molecular weight of less than about 1500 g/mol. Also, small molecules typically have multiple carbon-carbon bonds. Small molecules can be used as test compounds in the inventive screening method. In one embodiment, small molecules do not exclusively comprise peptide (amide) bonds. In another embodiment, small molecules are not oligomeric. Exemplary small molecule compounds include, but are not limited to, peptidomimetics, small organic molecules (e.g., Cane et al. 1998. Science 282:63; incorporated herein by reference), and natural product extract libraries. In another embodiment, the compounds are small, organic non-peptidic compounds. In a further embodiment, a small molecule is not biosynthetic. For example, a small molecule is preferably not itself the product of transcription or translation.

“Spliced forms of XBP-1”: As used herein, the term “spliced forms of XBP-1” refers to the spliced, processed form of the XBP-1 mRNA or the corresponding protein. Spliced forms of XBP-1 are key factors in the transcriptional regulation of molecular chaperones and enhance compensatory UPR. Activation of UPR leads to activation of IRE-1 which has an endoribonuclease activity which generates the active (i.e., spliced) form of XBP-1. Spliced XBP-1 is involved in the transcription of ER chaperones and components of the ER associated degradation (BRAD) pathway. In murine and human cells, a 26 nt intron is excised upon splicing of XBP-1 resulting in an increase in mobility of the mRNA when subject to electrophoresis. Spliced XBP-1 products can be detected by rtPCR, preferably quantitative rtPCR, using primers flanking the splice site for the PCR. The design of primers to detect relative quantities of unspliced and spliced mRNAs is well known to those skilled in the art. In mammalian cells, this splicing event results in the conversion of a 267 amino acid unspliced XBP-1 protein to a 371 amino acid spliced XBP-1 protein due to a frameshift in the coding sequence. The spliced XBP-1 then translocates into the nucleus where it binds to its target sequences to induce the transcription of molecular chaperones and other components of the UPR. An increase in the amount of spliced XBP-1 is preferably determined by an increase in the level of spliced XBP-1 as compared to the level of unspliced XBP-1. Unspliced XBP-1 is a negative regulator of the transcription of XBP-1.

“Subject”: The term “subject” refers to living organisms. In certain embodiments, the living organism is an animal. In certain preferred embodiments, the subject is a mammal. In certain embodiments, the subject is a domesticated mammal. In certain embodiments, the subject is a human. Examples of subjects include humans, monkeys, dogs, cats, mice, rats, cows, horses, goats, and sheep. The subject may be diagnosed with tuberous sclerosis. In other embodiments, the subject has been diagnosed with some other hamartomatous disease.

“Treat”: As used herein, the term “treat” means to care for or deal with medically, or to act upon with some agent especially to improve or alter a condition or disease state such as tuberous sclerosis or other hamartomatous disease with an effective amount of an ER stress modulating agent. Treatment need not be curative. More than one dose of an agent that modulates ER stress may be required for the treatment of disease.

“Tuberous sclerosis”: As used herein, the term “tuberous sclerosis” refers to the complex of signs and symptoms associated with tuberous sclerosis complex. In certain embodiments, the signs and symptoms are a result of defects in the genes TSC1 or TSC2. Tuberous sclerosis can lead to tumors in any organ of the body including kidneys, heart, eyes, lungs, pancreas, liver, and skin. The disease may also lead to cysts such as bone cysts or kidney cysts. Tuberous sclerosis is frequently associated with neurological problems such as seizures and behaviors problems. Treatment of tuberous sclerosis refers to reducing any of these signs or symptoms including reducing tumor burden, reducing development of tumors, reducing number of tumors, reducing frequency or severity of seizures; reducing the number or frequency of skin lesions, improving renal function, etc.

### DETAILED DESCRIPTION AND PREFERRED EMBODIMENTS

Endoplasmic reticulum (ER) stress has been found to be important in the pathogenesis of a variety of diseases including α1-anti-trypsin deficiency, urea cycle disorders, type I and type II diabetes, obesity, insulin resistance, and cystic fibrosis. The present invention stems from the recognition that ER stress is implicated in the pathogenesis of tuberous sclerosis and other hamartomatous diseases.

In particular, cells deficient in either TSC1 or TSC2, the genes implicated in tuberous sclerosis, have been shown to have increased PERK and S6K1 phosphorylation, increased spliced XBP-1 levels, and increased GRP78/BIP and CHOP mRNA levels as compared to wild type cells in response to glucose starvation. The increase in these markers of ER stress was blocked by rapamycin which inhibits signaling through the mTOR pathway, one of the major sensors of nutrients and energy balance in the cells. PERK phosphorylation was also blocked by treatment of the cells with cyclohexamide. Cyclohexamide inhibits protein synthesis, thereby decreasing ER load, which in turn decreases ER stress in response to glucose starvation. Furthermore, hemangiomas from liver and cystic adenomas from kidney of TSC2+/− mice exhibit signs of ER stress, particularly, increased IRE-1, PERK, and eIF2-α phosphorylation in liver hemangiomas, and increased GRP78/BIP expression in renal adenomatas tissue, but not in the adjacent normal tissue. Similar increases in ER stress markers, including increased eiF2α and increased expression of GRB78/BIP protein were observed in a study using human tuber dissected in an epilepsy surgery. An increase in S6 phosphorylation, indicating the hyperactivity of the mTOR pathway was observed. Therefore, tissue samples from mouse and human tumors confirm the observations made in MEF regarding ER stress in TSC-deficient cells.

Incubating cells deficient in TSC1 or TSC2 with a chemical chaperone such as PBA alleviates ER stress as demonstrated by a significant decrease in PERK, s6K1, and s6 phosphorylation, and in XBP-1 mRNA splicing. These data demonstrate chemical chaperones are effective in reducing cell stress as demonstrated by a reduction in the levels of multiple ER stress markers.

Loss of TSC1 or TSC2 function leads to severe inhibition of insulin/IGF-1 stimulated insulin responsive substrate (IRS)-1 and -2 tyrosine phosphorylation, and distally Akt phosphorylation. Moreover, UPR leads to an inhibition of insulin receptor signaling. Treatment of both TSC1- and TSC2-deficient cells with PBA resulted in a correction of insulin signaling as demonstrated by phosphorylation of IRS-1, IRS-2, and Akt in response to insulin stimulation. Further, ER stress was shown to promote degradation of IRS-1. Cells were treated with ER stress inducers thapsigargin or tunicamycin in the presence of a proteosome inhibitor, either epoxomicin or MG132. IRS-1 was found to be extensively ubiquitinated and targeted for protein degradation. Ubiquitination could be blocked by preincubation of the cells with a JNK inhibitor. These data provide a possible mechanism for disruption if insulin signaling in cells undergoing ER stress, and provide a method for restoring insulin signaling in cells undergoing ER stress using a chemical chaperone.

TSC-deficient cells were also found to be sensitive to ER stress inducers (i.e., thapsigargin or tunicamycin) as exhibited by increased production of spliced XBP-1 and cell death at low concentrations of ER stress inducers that had no effect on wild type cells at the same concentration. TSC2-deficient cells were at least partially rescued from ER stress inducer sensitivity by transfection with a retroviral expression vector encoding TSC2, or by treatment with the chemical chaperone PBA.

The utility of low levels of ER stress inducing agents to promote killing of TSC-deficient cells was confirmed in a mouse study. Animal studies in approximately one year old heterozygous TSC2+/− mice demonstrated that this increased sensitivity to ER stress could be exploited for the treatment of tuberous sclerosis. ER stress inducing agents were administered to heterozygous TSC2+/− mice with both kidney adenomas and liver hemangiomas. An apoptosis was observed in the tumors of the mice. Therefore, TSC-deficient cells such as those found in tumors of subjects suffering from tuberous sclerosis can be selectively killed by ER stress inducing agents. No overt toxicity was observed in normal tissue.

Based on the role of ER stress in the pathogenesis of tuberous sclerosis, the present invention includes the use of agents that modulate ER stress in the treatment of tuberous sclerosis. Any agent known to reduce or modulate ER stress can be useful in treating tuberous sclerosis. In certain embodiments, these agents act to reduce or prevent ER stress. In certain embodiments, the agent may increase the capacity of the ER to process proteins (e.g., increasing the expression of ER chaperones, increasing the levels of post-translational processing machinery). In other embodiments, the agent may reduce the quantity of proteins to be processed by the ER (e.g., decreasing the total level of protein produced in a cell, reducing the level of protein processed by the ER, reducing the level of mutant proteins, reducing the level of misfolded proteins). Yet other agents may cause the release of misfolded/mutant proteins from the ER. The agent may work in all cells, or the effect may be limited to certain cells type (e.g., secretory cells, epithelial cells, hepatocytes, adipocytes, endocrine cells, etc.). In certain embodiments, the agents are particularly useful in reducing ER stress in the cells of tuberous sclerosis tumors (e.g., tumors formed due to a TSC-deficiency regardless of the tissue affected). In other embodiments, the agents are useful in reducing ER stress in TSC-deficient cells. In other embodiments, agents to induce ER stress may be administered initially to reduce or eliminate the tumor burden, either alone or in conjunction with surgery. Upon elimination of the tumor(s), agents to decrease ER stress (e.g., chemical chaperones) can be administered to prevent recurrence of tumors. The agents may work on the transcriptional, translational, post-translational, or protein level to reduce or prevent ER stress.

The administration of an effective dose of an ER stress modulator, or a combination therapy including an ER stress modulator, to a subject to alleviate, prevent, and/or treat tuberous sclerosis can reduce at least one sign or symptom of the disease, reduce the consequences of the disease, reduce the development of tuberous sclerosis-associated tumors, or provide some other transient beneficial effect to the subject. The invention includes the use of such agents for the preparation of a medicament for the alleviation, prevention, and/or treatment of tuberous sclerosis or other hamartomatous diseases. In certain embodiments, the inventive treatments and medicaments reduce levels of ER stress markers in cells (e.g., adipocytes, hepatocytes) or tuberous sclerosis tumors.

In other embodiments, ER stress modulating agents act to increase ER stress resulting in the death of TSC-deficient cells. Examples of agents that induce ER stress include, but are not limited to, thapsigargin, tunicamycin, and azetidine-2 carboxylic acid (Azc). These agents are particularly useful in controlling the tumors associated with tuberous sclerosis. The invention includes the use of such agents for the preparation of a medicament for the alleviation, prevention, and/or treatment of tuberous sclerosis or other hamartomatous diseases. As shown in herein, the tumors associated with tuberous sclerosis are deficient in TSC and are therefore sensitive to ER stress induced death. Contacting TSC-deficient cells with an ER stress inducing agent results in killing these cells while sparing cells in the surrounding normal tissue despite the toxicity of many of the agents at higher doses. Therefore, the present invention provides a medicament and a method for controlling tumors of tuberous sclerosis by administering a therapeutically effective amount of an ER stress inducing agent to a subject.

In certain embodiments, the ER stress modulating agent is a small molecule. Particularly useful agents are known as chemical chaperones, which are known to stabilize proteins against denaturation and/or promote proper folding of both wild type and mutant proteins thereby preserving the protein's structure and function. The agent may be any type of chemical compound. The agent may be a small molecule, organometallic complex, an inorganic compound, a protein, a glycoprotein, a peptide, a carbohydrate, a lipid, or a nucleic acid. Chemical chaperones include glycerol, D₂O, dimethylsulfoxide (DMSO), 4-phenyl butyrate (PEA), tauroursodeoxycholic acid (TUDCA), ursodeoxycholic acid (UDCA), glycine betaine (betaine), glycerolphosphocholine (GPC), methylamines, and trimethylamine N-oxide (TMAO). In certain embodiments, combinations of one or more chemical chaperones may be used. These chemical chaperones are administered in doses ranging from 10 mg/kg/day to 10 g/kg/day, preferably 100 mg/kg/day to 5 g/kg/day, more preferably from 500 mg/kg/day to 3 g/kg/day. In certain embodiments, the agent is administered in divided doses (e.g., twice per day, three times a day, four times a day, five times a day). In other embodiments, the agent is administered in a single dose per day.

The agent to modulate ER stress may be combined with one or more other pharmaceutical agents, particularly agents traditionally used in the treatment of tuberous sclerosis to form a pharmaceutical composition or a medicament. Exemplary agents useful in combination with ER stress reducing agents include, but are not limited to, antineoplastic agents, anti-epileptic agents, vitamins, and minerals. In certain embodiments, the ER stress modulator is used in combination with rapamycin. In certain particular embodiments, PBA is combined with rapamycin. In certain embodiments, a chemical chaperone or ER stress modulator (e.g., PBA, TUDCA, UDCA, TMAO, or derivatives thereof) is used in combination with a vitamin, mineral, or other nutritional supplement.

In certain embodiments, the ER stress modulator (e.g., PBA, TUDCA, UDCA, TMAO, or derivatives thereof) is administered in a sub-clinical dose (e.g., an amount that does not manifest detectable therapeutic benefits when administered in the absence of a second agent). In such cases, the administration of such a sub-clinical dose of the ER stress modulator in combination with another agent results in at least an additive, preferably a synergistic effect. The ER stress modulator and other agent work together to produce a therapeutic benefit. In other embodiments, the other agent (i.e., not the ER stress modulator) is administered at a sub-clinical dose. In combination with an ER stress modulator, the combination exhibits a therapeutic effect. In yet other embodiments, both the ER stress modulator and the other agent are each administered in sub-clinical doses, and when combined the agents produce a therapeutic effect.

The dosages, route of administration, formulation, etc. for anti-neoplastic agents, anti-epileptic agents, vitamins, and minerals are known in the art. The treating physician or health care professional may consult such references as the Physician's Desk Reference (59th Ed., 2005), or Mosby's Drug Consult and Inreracations (2005) for such information. It is understood that a treating physician would exercise his or her professional judgment to determine the dosage regimen for a particular patient.

In certain embodiments, small molecule agents shown to reduce ER stress include 4-phenyl butyrate (PBA), tauroursodeoxycholic acid (TUDCA), ursodeoxycholic acid (UDCA), and trimethylamine N-oxide (TMAO). PBA is used currently to treat α1-anti-trypsin deficiency, urea cycle disorders, and cystic fibrosis. UDCA is used to treat primary biliary cirrhosis. Derivatives, salts (e.g., sodium, magnesium, potassium, magnesium, ammonium, etc.), prodrugs, esters, isomers, and stereoisomers of PBA, TUDCA, or TMAO may also be used to treat obesity, hypergylcemia, type II diabetes, and insulin resistance. Without wishing to be bound by any particular theory, these compounds are thought to work by allowing the ER to better tolerate misfolded and/or mutant proteins being processed by the ER.

In certain embodiments, a derivative of 4-phenyl butyrate useful in the present invention is of the formula:

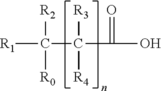

wherein n is 1 or 2;

R₀ is aryl, heteroaryl, or phenoxy, wherein the aryl, heteroaryl, and phenoxy being unsubstituted or substituted with, independently, one or more halogen, hydroxy, or lower alkyl (C₁-C₆) groups;

R₁ and R₂ are independently H, lower alkoxy (C₁-C₆), hydroxy, lower alkyl or halogen; and

R₃ and R₄ are independently H, lower alkyl, lower alkoxy or halogen; or

a pharmaceutically acceptable salt thereof; or a mixture thereof.

In certain embodiments, R₀ is a substituted or unsubstituted phenyl ring. In certain embodiments, R₀ is an unsubstituted phenyl ring. In other embodiments, R₀ is a monosubstituted phenyl ring. In yet other embodiments, R₀ is a disubstituted phenyl ring. In still other embodiments, R₀ is a trisubstituted phenyl ring. In certain embodiments, R₀ is a phenyl ring substituted with 1, 2, 3, or 4 halogen atoms. In certain embodiments, R₀ is a substituted or unsubstituted heteroaryl ring. In certain embodiments, R₀ is a naphthyl ring. In certain embodiments, R₀ is five- or six-membered ring, preferably a six-membered ring. In certain embodiments, R₁ and R₂ are both hydrogen. In certain embodiments, n is 1. In other embodiments, n is 2. In certain embodiments, both R₃ and R₄ are hydrogen. In other embodiments, at least one of R₃ or R₄ is hydrogen. In certain embodiments, the compound is used in a salt form (e.g., sodium salt, potassium salt, magnesium salt, ammonium salt, etc.). Other derivatives useful in the present invention are described in U.S. Pat. No. 5,710,178, which is incorporated herein by reference. 4-phenyl butyrate or its derivatives may be obtained from commercial sources, or prepared by total synthesis or semi-synthesis.

In certain embodiments, a derivative of TUDCA useful in the present invention is of the formula:

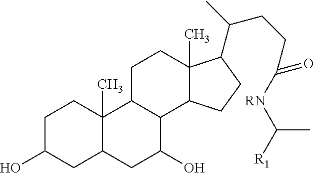

wherein:

R is —H or C₁-C₄ alkyl;

R₁ is —CH₂—SO₃R₃ and R₂ is —H; or R₁ is —COOH and R₂ is —CH₂—CH₂—CONH₂,

—CH₂—CONH₂, —CH₂—C14₂-SCH₃ or —CH₂—S—CH₂—COOH; and

R₃ is —H or a basic amino acid; or a pharmaceutically acceptable salt thereof. In certain embodiments, the stereochemistry of the derivative is defined as shown in the following structure:

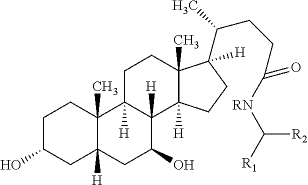

In certain embodiments, R is H. In other embodiments, R is methyl, ethyl, n-propyl, iso-propyl, n-butyl, iso-butyl, or tert-butyl, preferably, methyl. In certain embodiments, R₁ or R₂ is hydrogen. In certain embodiments, R₁ is —CH₂—SO₃R₃ and R₂ is —H. In other embodiments, R₁ is —COOH and R₂ is —CH₂—CH₂—CONH₂, —CH₂—CONH₂, —CH₂—CH₂—SCFI₃ or —CH₂—S—CH₂—COOH. In certain embodiments, R₃ is hydrogen. In certain embodiments, R₃ is lysine, arginine, ornithine, or histidine. Derivatives of TUDCA and ursodeoxycholic acid (UDCA) may be obtained from commercial sources, prepared from total synthesis, or obtained from a semi-synthesis. In certain embodiments, the derivative is prepared via semi-synthesis, for example, as described in U.S. Pat. Nos. 5,550,421 and 4,865,765, each of which is incorporated herein by reference.

In certain embodiments, derivative of trimethylamine N-oxide useful in the present invention is of the formula:

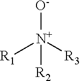

wherein:
R₁, R₂, and R₃ are independently hydrogen, halogen, or lower C₁-C₆ alkyl; or a pharmaceutically-acceptable salt thereof; or a mixture thereof. In certain embodiments, R₁, R₂, and R₃ are the same. In other embodiments, at least one of R₁, R₂, and R₃ is different. In yet other embodiments, all of R₁, R₂, and R₃ are different. In certain embodiments, R₁, R₂, and R₃ are independently hydrogen or lower C₁-C₆ alkyl. In yet other embodiments, R₁, R₂, and R₃ are independently lower C₁-C₆ alkyl. In still other embodiments, R₁, R₂, and R₃ are independently methyl, ethyl, or propyl. In certain embodiments, R₁, R₂, and R₃ are ethyl. Derivatives of TMAO may be obtained from commercial sources, or prepared by total synthesis or semi-synthesis.

In treating an animal, suffering from tuberous sclerosis, a therapeutically effective amount of the agent is administered to the subject via any route to achieve the desired biological result. Any route of administration may be used including oral, parenteral, intravenous, intraarterial, intramuscular, subcutaneous, rectal, vaginal, transdermal, intraperitoneal, and intrathecal. In certain embodiments, the agent is administered parenterally. In other embodiments, the agent is administered orally.

In the use of PBA, TUDCA, UDCA or TMAO, the agent is preferably administered orally; however, any of the administration routes listed above may also be used. In certain embodiments, the PBA, TUDCA, UDCA or TMAO is administered parenterally, PBA is administered in doses ranging from 10 mg/kg/day to 5 g/kg/day, preferably from 100 mg/kg/day to 1 g/kg/day, more preferably from 250 mg/kg/day to 750 mg/kg/day. TUDCA is administered in doses ranging from 10 mg/kg/day to 5 g/kg/day, preferably from 100 mg/kg/day to 1 g/kg/day, more preferably from 250 mg/kg/day to 750 mg/kg/day. TMAO is administered in doses ranging from 0.1 g/kg/day to 10 g/kg/day, preferably from 0.5 g/kg/day to 5 g/kg/day, more preferably from 500 mg/kg/day to 2.5 g/kg/day. In certain embodiments, the agent is administered in divided doses (e.g., twice per day, three times a day, four times a day, five times a day). In other embodiments, the agent is administered in a single dose per day.

### Pharmaceutical Compositions and Medicaments

Pharmaceutical compositions and medicaments of the present invention may include a pharmaceutically acceptable excipient or carrier. As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

The pharmaceutical compositions of the invention may be provided in a kit with other agents used to treat tuberous sclerosis or other hamartomatous disease. The kit may include instructions for the treating physician and/or patient, which may include dosing information, safety information, list of side effects, chemical formula of agent, mechanism of action, etc. In certain embodiments, the kit may include materials for administering the pharmaceutical composition. For example, the kit may include a syringe, needle, alcohol swabs, etc. for the administration of an injectable preparation. In certain embodiments when two or more agents are provided in a kit, the active pharmaceutical ingredients may be formulated separately or together. For example, the kit may include a first container with as ER stress modulator (e.g., PBA, TUDCA, UDCA, TMAO, or a derivative thereof) and a second container with a second agent used in treating tuberous sclerosis (e.g., anti-neoplastic agents, anti-epileptic agents, vitamins, and minerals, as described above). In certain embodiments, the active pharmaceutical ingredients are formulated separately. In other embodiments, the active pharmaceutical ingredients are formulated together.

### Screening for ER Stress Reducers

As demonstrated herein, ER stress has been identified as a target for the treatment of tuberous sclerosis and other hamartomatous diseases. Markers of ER stress may be used as indicators of the disease or indicators of the effectiveness of treatment. With the need for new pharmaceutical agents that modulate ER stress, a method of identifying agents useful in the treatment of tuberous sclerosis is needed. Particularly, useful in a system for identifying agents as ER stress modulators are TSC-deficient cells. TSC1- and TSC2-deficient cells represent a cell model of spontaneous ER stress and provide a useful platform to investigate ER stress in the cell. These TSC-deficient cells are useful in screening for ER stress modulators without the use of compounds or drugs that modify ER function.

Such cells can be incorporated into a kit to allow for the screening of ER stress reducers. Kits can further include control agents known to increase (e.g., ER stress inducers) or decrease ER stress (e.g., chemical chaperones), tissue culture reagents such as glucose-free media. The kit may include primers, hybridization probes, polynucleotides, antibodies, antibody fragments, gels, buffers, enzyme substrates, ATP or other nucleotides, tools for obtaining cells or a biopsy from the subject, instructions, software, etc. These materials for performing the diagnostic method may be conveniently packaged for use by a physician, scientist, or other individual skilled in the art.

In certain embodiments, a test compound or a collection of test compounds is assayed using TSC-deficient cells or whole animals (e.g., heterozygous TSC1−/+ or TSC2−/+ animals, or mosaic homozygous TSC1−/− or TSC2−/− animals) to identify compounds that reduce or modulate ER stress in vivo or in vitro, preferably in vivo. These test compounds may be any type of chemical compound including small molecules, proteins, peptides, polynucleotides, carbohydrates, lipids, natural products, etc. In certain embodiments, a collection of compounds is screened using a method of the invention. In a preferred embodiment, a collection of compounds is screened using a high throughput screening method.

The test compounds can undergo preliminary screenings in one or more in vitro assays to identify compounds that modulate at least one marker of ER stress. The compounds can then be tested in appropriate animal models, such as the TSC-deficient heterozygous mice, using routine methods to determine if the test compound is also effective in vivo. Such methods of identification of compounds in vitro for further analysis in vivo is frequently employed in drug identification screening methods.

A variety of test compounds can be evaluated using the screening assays described herein. The term “test compound” includes any agent that is employed in the inventive screening system and assayed for its ability to modulate (i.e., increase or decrease) ER stress. More than one compound, e.g., a plurality of compounds, can be tested at the same time for their ability to modulate ER stress in the inventive system. Preferably, the subject assays identify compounds not previously known to have the effect on ER stress, although the compound may be well known. In one embodiment, high throughput screening techniques and apparatuses can be used to identify compounds that modulate ER stress. Many methods of high throughput screening are well known to those skilled in the art. The exact method of screening test compounds is not a limitation of the instant invention.

In certain embodiments, the compounds to be tested can be derived from libraries (i.e., are members of a library of compounds). These collections may be historical libraries of compounds from pharmaceutical or biotech companies. In certain embodiments, the collection may be a combinatorial library of chemical compounds. The collection may include at least about 5, 10, 50, 100, 500, 1000, 10000, 100000, or 1000000 compounds. While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909; each of which is incorporated herein by reference) peptoids (Zuckermann (1994). J. Med. Chem. 37:2678; incorporated herein by reference) oligocarbamates (Cho et al. (1993). Science. 261:1303; incorporated herein by reference), and hydantoins (DeWitt et al. supra; incorporated herein by reference). There libraries are merely examples of the types of libraries that can be prepared and are not meant to limit the types of libraries that can be screened using the inventive screening system. An approach for the synthesis of molecular libraries of diverse small organic molecules has been described (Carell et al. (1994). Angew. Chem. Int. Ed. Engl. 33:2059; Carell et. al. (1994) Angew. Chem. Int. Ed. Engl. 33:206 1; each of which is incorporated herein by reference).

The compounds of the present invention can be obtained using any of the numerous approaches in combinatorial library methods known in the art, including: biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, non-peptide oligomer, or small molecule libraries of compounds (Lam (1997) Anticancer Drug Des. 12:145; incorporated herein by reference). Other exemplary methods for the synthesis of molecular libraries can be found in the art, for example in: Erb et al., (1994). Proc. Natl Acad Sci. USA 91:11422; Horwell et al. (1996) Immunopharmacology 33:68; and in Gallop et al. (1994); J. Med Chem. 37:1233; each of which is incorporated herein by reference.

Libraries of compounds can be presented in solution (e.g., Houghten (1992) Biotechniques 13:412-421; incorporated herein by reference), or on beads (Lam (1991) Nature 354:82-84; incorporated herein by reference), chips (Fodor (1993) Nature 364:555-556; incorporated herein by reference), bacteria (Ladner, U.S. Pat. No. 5,223,409; incorporated herein by reference), spores (Ladner, U.S. Pat. No. 5,223,409; incorporated herein by reference), plasmids (Cull et al. (1992) Proc. Natl. Acad. Sci. USA 89:1865-1869; incorporated herein by reference) or on phage (Scott and Smith (1990) Science 249:386-390; incorporated herein by reference); (Devlin (1990) Science 249:404-406; incorporated herein by reference); (Cwirla et al. (1990) Proc. Natl. Acad Sci. 87:6378-6382; incorporated herein by reference); (Felici (1991) J. Mol. Biol. 222:301-310; incorporated herein by reference). In still another embodiment, the combinatorial polypeptides are produced from a cDNA library.

In the screening system, TSC-deficient cells are contacted with a test compound. In certain embodiments, the cells are deficient in TSC1. In certain embodiments, the cells are TSC1−/−. In other embodiments, the cells are TSC1−/+. In other embodiments, the cells are deficient in TSC2. In certain embodiments, the cells are TSC2−/−. In other embodiments, the cells are TSC2−/+. The TSC-deficiency in the cells may be the result of genetic engineering of the cells or animals. In other embodiments, the cells may be from a naturally occurring TSC-deficient cells from a cell line or tumor. The cells are preferably animal cells. In certain embodiments, mammalian cells are preferred, human cells. The cells may be derived from any organ system. In certain embodiments, cells are derived from tuberous sclerosis-associated tumors.

In screening for agents that reduce ER stress, the TSC-deficient cells are contacted with a test compound(s). Alternatively, cells stimulated with an agent to induce ER stress prior to contact with the compound. The level of ER stress markers may be assayed before and/or after addition of the test compound to determine if the compound modulates ER stress. A control is preferably used where no test compound is added to the cells. Time course assays can be used to further analyze test compounds. A positive control agent known to reduce ER stress (e.g., PBA) or that inhibits signaling through the mTOR pathway (e.g., rapamycin) can also be included. An additional control may also be used in the assay where an agent known to induce ER stress (e.g., thapsigargin, tunicamycin, azetidirie-2 carboxylic acid) is added to the cell. In certain embodiments, one ER marker is measured. In other embodiments, the levels of a combination of two, three, four, five, six, or more ER stress markers are measured. Test compounds that reduce the levels of ER stress markers by at least about 10%, 20%, 30%, 40%, 50%, 60%, 70%, 80%, 90%, 95%, 99%, or 100%, preferably at least about 25%, more preferably at least about 50%, are considered useful for evaluation as ER stress reducers in further in vitro and in vivo testing. Test compounds that increase the levels of ER stress markers by at least about 10%, 20%, 30%, 40%, 50%, 60%, 70%, 80%, 90%, 95%, 99%, or 100%, preferably at least about 25%, more preferably at least about 50%, are considered useful for evaluation of ER stress inducer in further in vitro and in vivo testing. As would be appreciated by one of skill in this art, the test compound may be tested at various concentrations and under various conditions (e.g., various cell types, various degrees of ER stress in the cell, various formulations, combined with other ER stress modulators).

In another aspect, the invention provides for a method of identifying compounds that prevent ER stress. In screening for compounds that prevent ER stress, the cells are not experiencing ER stress before they are contacted with the test compound. After the cells are contacted with the test compound, an agent known to cause ER stress is added to the cells, and then the level of at least one ER markers is measured to determine whether the compound is able to prevent ER stress. As would be appreciated by one of skill in this art, the test compound may be tested at various concentrations for various times and under various conditions and levels of ER stress markers. Test samples can be compared to appropriate control samples.

In another aspect, the invention provides for a method of identifying compounds that induce ER stress. In screening for compounds that induce ER stress, cells may or may not be experiencing ER stress prior to being contacted with the test compound. Cells not experiencing ER stress can be assayed for the presence of at least one marker of ER stress. Cells experiencing ER stress (e.g., TSC-deficient cells) can be assayed for apoptosis. Test compounds found to induce apoptosis can be retested in the presence of a compound that blocks mTOR signaling (e.g., rapamycin) to determine if apoptosis is due to increased ER stress. Alternatively, TSC-deficient cells can be screened in parallel with cells not experiencing ER stress (i.e., wild type cells), preferably cells of the same type (e.g., both fibroblast cell lines). Compounds that induce apoptosis in TSC-deficient cells, but not the wild type cells are likely inducing apoptosis by increasing ER stress.

Agents identified by the methods of the invention may be further tested for toxicity, pharmacokinetic properties, use in vivo, etc. so that they may be formulated and used in the clinic to treat tuberous sclerosis. The identified agents may also find use in the treatment of other diseases associated with ER stress.

### Screening, Diagnosing, and Monitoring the Progression of Tuberous Sclerosis Using ER Stress Markers

The realization of the role of ER stress in tuberous sclerosis and other hamartomatous diseases allows for the diagnosis of conditions associated with ER stress, the screening of subjects at risk for developing conditions associated with ER stress, the following of the progression of the disease, and the following the effectiveness of the treatment. Tuberous sclerosis has been demonstrated herein to be associated with ER stress. Therefore, measuring the level of an ER stress marker(s) in a subject, preferably in effected tissue, allows for determining whether a patient has tuberous sclerosis. Determining the level of an ER stress marker may also be used to follow the progression of a patient's tuberous sclerosis or to follow the effectiveness of the treatment of a patient's disease. Tissue can be obtained by biopsy, surgical resection, or other methods well known to those skilled in the art.

ER stress markers and methods for measuring them have been identified and are discussed herein. These ER stress markers may be measured using any techniques known in the art for measuring mRNA levels, protein levels, protein activity, or phosphorylation status. Exemplary techniques for measuring ER stress markers include western blot analysis, ELISA, northern blot analysis, immunoassays, quantitative PCR analysis, and enzyme activity assay (for a more detailed description of these techniques, please see Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989); each of which is incorporated herein by reference). The levels of ER stress markers may be determined from any cells in the subject's body. In certain embodiments, the cells are cells from a tuberous sclerosis associated tumor. The cells may be obtained in any manner including biopsy or surgical excision.

In following disease progression or effectiveness of treatment, one may determine the levels of one, two, three, four, five, or six ER stress markers. In certain embodiments, the level of only one ER stress marker is determined. In other embodiments, the levels of at least two ER stress markers are determined. In yet other embodiments, the levels of at least three ER stress markers are determined.

The invention also provides kits and systems for measuring the levels of various ER stress markers in a subject with tuberous sclerosis. The kit may include primers, hybridization probes, polynucleotides, antibodies, antibody fragments, gels, buffers, enzyme substrates, ATP or other nucleotides, tools for obtaining cells or a biopsy from the subject, instructions, software, etc. These materials for performing the diagnostic method may be conveniently packaged for use by a physician, scientist, pathologist, nurse, lab technician, or other health care professional.

### Other Hamartomatous Diseases

Besides tuberous sclerosis, there are other hamatomatous diseases. These disease may also be associated with ER stress, and therefore be susceptible to treatment with ER stress modulators. Other hamartomatous diseases include pulmonary hamartoma, von Meyenburg complex, proteus syndrome, Birt-Hogg-Dube syndrome, multiple hamartoma syndrome, neurofibromatosis type I, Peutz-Jeghers syndrome, Riley-Smith syndrome, and angiomyolipoma. In certain embodiments, the disease are treated with ER stress reducers described herein. ER stress markers may be used to monitor the progression of the disease or the effectiveness of treatment.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Biochemical Reagents

Anti-MS-1 and anti-IRS-2 antibodies were obtained from Upstate Biotechnology (Charlottesville, Va.). Antibodies against phosphotyrosine, eIF2α, and insulin receptor β subunit were from Santa Cruz Biotechnology (Santa Cruz, Calif.). Anti-phospho S6K, anti-S6K1, anti-phospho-PERK, antiphospho-eIF2α, anti-Akt and anti-phospho-Akt antibodies, and c-Jun recombinant protein were from Cell Signaling Technology (Beverly, Mass.). Fluorescein-conjugated (FITC-conjugated) goat anti-rabbit IgG were from Jackson Immuno Research Laboratories (West Grove, Pa.). Thapsigargin was from Calbiochem (San Diego, Calif.). Cell Death Elisa Kit and BM Chemiluminescence Blotting Substrate (POD) were from Roche (Indianapolis, Ind.). The antiphospho-IRE-1 antibody was a gift from Dr. Fumihiko Urano from University of Massachusetts

### Example 2

### Analysis of ER Stress Parameters

All of the mouse embryonic fibroblast (MEF) cell lines (TSC1+/+, TSC1−/−, TSC2+/+, TSC2−/−) were cultured in medium containing DMEM-H+10% fetal bovine serum (FBS)+1% PS (penicillin-streptomycin complex) in 175 cm² cell culture flasks using standard methods. Upon reaching 90% confluency, cells were split into 10 cm dishes at 30-40% confluency and grown again in DMEM-H+10% FBS+1% PS to about 60% confluency for experiments.

At the start of the experiment, cells were placed in fresh media and treated with 200 nM Rapamycin or 10 mM PSA, and DMSO or PBS as respective vehicle controls (PBA was dissolved in PBS, and rapamycin was dissolved in DMSO), for 19 hours. As a vehicle control, DMSO was not present at a sufficiently high concentration to act as a chemical chaperone. Cells were then washed 3 times with serum-free DMEM-H+1% PS and incubated for 5 hours in serum free DMEM-H+1% PS in the presence of rapamycin or PBA and their appropriate vehicle controls. At the end of the incubation, the media was removed and dishes were immediately frozen in liquid nitrogen and stored at −80° C. untile further use.

Protein extracts were prepared with a lysis buffer containing 25 mM Tris-HCl (pH 7.4), 2 mM Na₃VO₄, 10 mM NaF, 10 mM Na₄P₂O₇, 1 mM EGTA, 1 mM EDTA, 1% NP-40, 5 ug/ml leupeptin, 5 ug/ml aprotinin, 10 nM okadaic acid, and 1 mM phenylmethylsulfonyl fluoride (PMSF). Immunoprecipitations and immunoblotting experiments were performed with 200 and 75 μg total protein, respectively without any freeze-thaw cycles from individual aliquots.

### Example 3

### Western Blotting

Protein concentrations were normalized and the desired amounts were aliquotted into tubes. Laemelli buffer was added to a 1× final concentration and the samples were boiled for five minutes. After boiling, the samples were incubated at room temperature for 20 minutes. The boiled, cooled lysates were centrifuged at 14,000 rpm and subject to SDS-PAGE. Proteins were transferred to PVDF membranes for western blotting.

Membrane blotting was performed using standard techniques and reagents. Appropriate modification depending on the antibodies used and other considerations, is within the ability of those skilled in the art. Membranes were blocked in 10% blocking reagent for 1 hour prior to exposure to primary antibody in tris-buffered saline-tween (TBST), pH 7.4, overnight at 4° C. Following overnight incubation, membranes were washed in TBST for 3×20 minutes and placed into secondary antibody for 1 hour. Subsequently the membrane was washed 3×20 minutes in TBST. After the washing period, the membranes were developed by using a chemiluminescense kit and the bands were visualized using a phospho imager system (VersaDoc Imaging System, Model3000).

### Example 4

### Immunoprecipitation

Cell lysates were prepared as above, and subsequently incubated with primary antibody and Sepharose beads, either Protein A Sepharose or Protein G Sepharose depending on the antibody. The samples were incubated on a rotating apparatus overnight at 4° C. Subsequently, beads were centrifuged at 14,000 rpm and washed 3 times with cold lysis buffer. If immunoprecipitates were to be subjected to SDS-PAGE, they were boiled in 2× Laemmli buffer for 5 minutes prior to loading.

### Example 5

### c-Jun Amino Terminal Kinase (JNK) Kinase Assay

Following immunoprecipitation the beads were washed 3 times with lysis buffer as described above, and two times with JNK kinase assay buffer (25 mM HEPES (pH: 7.4), 20 mM MgCl₂, 20 mM β-glycerophosphate, 0.5 mM EGTA, 0.5 mM NaF, 0.5 mM NaOrthoVanadate, 1 mM PMSF) for equilibration. After washing with kinase buffer, the beads were incubated in 17 μl kinase buffer, 1 μl [³²P]γ-ATP and 4 μg of c-Jun fusion protein at 30° C. for 20 minutes. The reaction was terminated by the addition of Laemmli buffer. The samples were resolved by SDS-page and transferred to a PVDF membrane as described above. Phosphorylated c-Jun bands were visualized prior to western blotting.

### Example 6

### TSC2- and TSC1-Deficient Cells have Increased Levels of ER Stress Markers

Phosphorylation state of the ER stress markers PERK and s6K1 were analyzed in both wild type (TSC2+/+ and TSC1+/+) and TSC-deficient (TSC2−/− and TSC1−/−) MEFs. Cells were cultured and treated with DMSO (vehicle control) or rapamycin as discussed above. Rapamycin inhibits signaling through the mTOR pathway which is required for ER stress response. The DMSO was present at too low of a concentration to act as a chemical chaperone. Cells were harvested and protein and RNA were isolated. Protein samples were resolved by SDS-PAGE and proteins were transferred to PVDF membrane for western blotting as described above. RNA was subjected to rt-PCR using probes designed to mouse XBP-1 to detect splicing.

The western blot was probed with antibodies targeted to phosphorylated (i.e., activated) ER stress markers PERK, s6K1, and ribosomal protein S6, and to total s6K1 as a control for protein loading. The levels of unspliced (XBP-1(u)) and spliced (XBP-1(s)) mRNA were also analyzed, as were the levels of GRP78/BIP and CHOP. MEFs deficient in either TSC1 or TSC2 demonstrated increased PERK, s6K1, and ribosomal protein S6 phosphorylation compared to wild type (TSC1+/+ and TSC2+/+) control cells, reflecting increased ER stress or unfolded protein response (UPR) in the absence of the TSC1 or TSC2 gene. An increase in p70 s6K1 kinase activity is known to occur with ER stress and was used here as a positive control. Incubation with rapamycin (200 nM, 12 hours), which blocks mTor activity, abolished the PERK, s6K1, and S6 phosphorylation and substantially reduced the presence of spliced XBP-1. These data demonstrate that ER stress is induced by glucose starvation of TSC-deficient cells and that ER stress response requires signaling through the mTOR pathway.

The ER stress response to glucose starvation in TSC2−/− cells is a result of the deletion of TSC2. An expression vector with (+TSC2) or without (+VEC) was transfected into the TSC2−/− MEFs prior to serum starvation. Expression of TSC2 from the retroviral vector substantially decreased the ER stress response as shown by a decrease in the spliced XBP-1 product and decreased phosphorylation of PERK, S6K1, and S6.

ER stress causes an increase in protein translation, at least partially due to induction of the UPR. To investigate the role of increased translation initiation and consequently upregulated protein synthesis, MEFs undergoing ER stress were treated with cycloheximide, a well known inhibitor of translation initiation. Cyclohexamide was found to decrease phosphorylation of PERK in both TSC1- and TSC2-deficient cells. These data demonstrate that decreasing ER load results in a decrease in the ER stress induced by TSC-deficiency.

These data demonstrate an increase in ER stress due to loss of TSC1 or TSC2 can be inhibited by blocking signaling through the mTOR pathway. ER stress can also be attenuated by expression of TSC2 in TSC2−/− cells. Moreover, inhibition of protein synthesis also results in a decrease in the presence of ER stress markers in TSC-deficient cells.

### Example 7

### Tissue Preparation and Immunofluoresence Staining of Liver Hemangiomas and Adenomas from TSC2-Deficient Mice

TSC2+/− mice develop several tumors around 6-12 months due to a loss of heterozygosity (LOH). Frequently, giant hemangiomas develop in the liver and cystic adenomas develop in the kidney. Twelve month old TSC2+/− mice on a C57BL/6j-129/SvJae mix background were euthanized according to approved protocols. Livers and kidneys were dissected and directly fixed in 10% buffered neutral formalin. Following fixation the tissues were paraffin embedded, and 5 micron sections were prepared for hematoxylin and eosin (H&E) histological staining and imuunohistochemical analysis.

Briefly, the 5 micron sections were subjected to routine deparrafinization/hydration process. H&E staining was carried out using routine methods. Hematoxylin stains negatively charged nucleic acids (nuclei & ribosomes) blue, and eosin stains proteins pink to reveal cell morphology. Sections for immunofluorescence staining were washed 3×10 minutes in phosphate buffered saline (PBS), and incubated in 1% Triton X-100 in PBS for 10 minutes. Next, the samples were washed for 3×10 minutes in PBS, and incubated in 5% bovine serum albumin (BSA) in PBS for 1 hour. Following blocking, primary antibody incubation was performed overnight (about 14 hours) at 4° C. in 5% BSA in PBS. Sections were then washed 4×10 minutes and subjected to secondary blocking in 5% goat serum. Sections were then incubated with a FITC-conjugated secondary antibody for 60 minutes in the dark. At the end of secondary antibody incubation, sections were washed 3×10 minutes in the dark, overlaid with anti-fade reagent, and cover slips were applied.

### Example 8

### Tuberous Sclerosis Tumors from TSC-Deficient Mice Exhibit Increased ER Stress Markers

mTOR pathway is activated in the tumors in TSC-2+/− mice arising due to LOH. H&E stained tissue sections from liver hemangiomas and kidney adenomas that developed in TSC-2+/− mice around one year of age. Normal and tumor tissue are indicated. Immunofluorescent staining revealed that 56 phosphorylation in both liver and kidney tumors was increased, indicating signaling through the mTOR pathway. Analysis ER stress markers PERK, eIF2a, and IRE-1 phosphorylation showed a striking increase in the hemangiomas. GRP-78 protein levels were found to increase a significantly increase in kidney adenomas.

### Example 9

### Tuberous Sclerosis Tumors from Human Exhibit Increased ER Stress Markers

To confirm the results seen in tissue culture and mouse studies, and to extend findings to a new tumor type, a human tuber which was dissected in an epilepsy surgery from a 3 year old tuberous sclerosis and epilepsy patient was analyzed for the presence of ER stress markers. The enlarged glial cells (vimentin positive cells) had increased S6 phosphorylation, which indicates the hyperactivity of mTOR pathway. Analysis of eIF2a phosphorylation in the sections from the tuber revealed that enlarged glial cells have increased phosphorylation of eIF2α Enlarged neurons (SM-311 positive cells) also displayed up-regulation of eIF2α phosphorylation. An increase in GRP78/BIP protein levels was also observed. Taken together, the data strongly implicates that a loss of TSC function leads to development of perturbations in ER system and activation of UPR, and formation of tuber sclerosis tumors.

### Example 10

### Increased ER Stress can be Inhibited by PBA in TSC1- and TSC2-Deficient Cells

Phosphorylation state of the ER stress markers PERK, eIF2α, cJun, JNK and s6K1, and the level of spliced XBP-1 were analyzed in both wild type and TSC-deficient MEFs. Cells were treated with PBS (vehicle control) or PBA (chemical chaperone) as discussed above. Cells were harvested, samples were resolved by SDS-PAGE, proteins were transferred to PVDF membrane, and western blots were performed as described above.

PERK phosphorylation is upregulated in TSC2-deficient cells. An increase in S6K1 and eIF2α phosphorylation was also observed. PBA treatment relieved ER stress and blocked PERK and eIF2α phosphorylation. Phosphorylation of S6K1 was not blocked by PBA treatment. In addition, the c-Jun Amino Terminal Kinase-1 (JNK-1) activity was also suppressed by PBA treatment.

A similar experiment was performed using wild type and TSC1-deficient cells. PBA treatment of the TSC1-deficient cells was able to block phosphorylation of PERK. A substantial decrease in eIF2α phosphorylation was also observed as was a decrease in the amount of XBP-1 spliced product. These data demonstrate that that ER stress in both TSC1- and TSC2-deficient cells is reversible by treatment with a chemical chaperone.

### Example 11

### Modification of Insulin Signaling in Cells Undergoing ER Stress

For insulin signaling experiments, cells were treated exactly the same way as for analysis of ER stress parameters. At the end of a 5-hour starvation period, cells were either stimulated with PBS or with 100 nM insulin for 5 minutes for analysis of ISR phosphorylation analysis, or 15 minutes for Akt phosphorylation analysis. Samples were harvested and subjected to western blotting as described above. Blots were probed with primary antibodies targeted to total and phosphorylated insulin receptor substrate (IRS)-1, and -2, and Akt, a downstream target in the insulin signaling pathway.

Loss of TSC-1 and TSC-2 function was found lead to severe inhibition of insulin/IGF-1-stimulated IRS-1 and IRS-2 tyrosine phosphorylation, and distally Akt serine phosphorylation. As UPR leads to inhibition of insulin receptor signaling, the contribution of UPR to development of the negative feedback loop to insulin/IGF-1 signaling in TSC-1−/− and TSC-2−/− cells was investigated. Stimulation of TSC-1−/− and TSC-2−/− cells with insulin showed a slight or no increase in IRS-1 and -2 tyrosine and Akt serine phosphorylations. PBA treatment improved insulin induced IRS-1 and -2 tyrosine phosphorylation, and also Akt phosphorylation even in the conditions of severe S6K1 activation.

Treatment with PBA, and consequently a decrease ER stress led to an increase in protein levels of IRS-1 and to a lesser extent IRS-2. Therefore, the possibility that ER stress leads to increased degradation of IRS-1 was investigated. It has been previously shown that when ER stress is induced acutely, IRS-1 is highly phosphorylated at serine 307 residue, and insulin induced tyrosine phosphorylation is blocked. However, prolonged exposure to ER stress created either by thapsigargin or tunicamycin treatment lead to degradation of IRS-1 as demonstrated herein. Stimulation of cells either tunicamycin or thapsigargin for 8 hours in the presence of a 26S proteosome inhibitor, epoxomicin, lead to severe ubiquitination of IRS-1. Similar results were obtained by using MG132, another potent proteosome inhibitor. Eight hour stimulation with thapsigargin in the presence of MG132 leads to ubiquitination of IRS-1. This effect was blocked by preincubating the cells with JNK inhibitor, showing that JNK activity plays a pivotal role in degradation of IRS-1 during ER stress. As mentioned above, IRS-1 degradation was increased in both TSC1- and TSC2-deficient cells. Considering the fact that ER stress leads to degradation of IRS-1, and is up-regulated in TSC deficiency, the degradation pattern of IRS-1 and -2 after cycloheximide addition, with or without pretreatment with PBA, in TSC-1 and -2 deficient cells was examined. IRS-1 protein levels became totally undetectable in an hour after cycloheximide addition, whereas pretreatment with PBA extends this period up to 4 hours. Similar results were also obtained from TSC-2−/− cells, which strongly indicates that ER stress plays an important role in enhanced degradation of IRS-1 in TSC deficiency and, over all, contributes to the development of negative feed back loop for insulin resistance.

### Example 12

### TSC-1-Deficient Cells have Increased Sensitivity to ER Stress Inducing Agents

ER stress can cause apoptosis. The ability of ER stress inducers to promote apoptosis in TSC1-deficient cells at concentrations that do not promote apoptosis in normal cells was analyzed. Wild-type and TSC1−/− cells were plated in 96 well plates in DMEM-H+10% FBS+1% PS at 40-50% confluence. Upon reaching about 80% confluence, the cells were washed with DMEM-H without FBS and treated with the ER stress inducer thapsigargin (10 nM) or DMSO (vehicle control) for 6 hours in serum-free DMEM-H+1% PS. Apoptosis was analyzed by using Cell Death Elisa Kit using the manufacturer's instructions, or by detection of caspase 3 or PARP cleavage.

TSC-deficient cells and their corresponding control cells were exposed to extremely low doses of thapsigargin (0.05 nM) and tunicamycin (0.002 ug/ml) at which the UPR is not activated in wild type control cells. TSC1- and TSC2-deficient cells responded to the ER stress inducers by activating the UPR as determined by increased splicing of XBP-1 mRNA when compared with their controls. These data show that TSC-deficient cells are highly sensitive to ER stress inducing agents and respond with UPR activation.

TSC-deficient cells were then analyzed for apoptosis levels after thapsigargin and tunicamycin treatment. Induction of ER stress with thapsigargin in both TSC1−/− and TSC2−/− cells, lead to massive apoptosis after 6 hours, whereas no apoptosis was observed in control cells during the same time period. Biochemical analysis of apoptosis indicators such as cleaved caspase-3 or PARP cleavage was significantly induced after thapsigargin treatment in TSC1−/− cells when compared with their controls. Induction of ER stress also increased PARP and caspase-3 cleavage in TSC-2−/− cells which could be blocked either by reconstitution of TSC-2 deficient cells by expression of TSC2 from a retroviral expression vector, or rapamycin treatment to inhibit mTOR signaling.

The ability of TSC deficient cells to respond to glucose starvation in a similar way that they respond to ER stress inducing agents was investigated. Glucose starvation for 10 hours was not enough to induce XBP-1 splicing in wt cells; however, most of the XBP-1 mRNA were spliced in TSC-1 deficient cells after glucose starvation, indicating that TSC-1 deficient cells are much more sensitive to glucose starvation induced development of ER stress. The same is also true for TSC-2 deficient cells. The effect of 4-PBA on XBP-1 mRNA splicing after glucose starvation was analyzed. XBP-1 mRNA splicing was significantly reduced when the TSC-1/2 deficient cells are glucose starved in the presence of 4-PBA. CHOP expression was found to be up-regulated by ER stress and is an important element of ER stress induced apoptosis. Glucose starvation induced CHOP transcription was more than 10 fold (p<0.001) in TSC-1 deficient cells when compared with their controls, and 4-PBA treatment significantly reduced glucose starvation induced CHOP transcription. PARP and caspase-3 cleavage after glucose starvation with or without 4-PBA in TSC1 and TSC2-deficient cells was also investigated. Glucose starvation induced PARP and caspase-3 cleavage was blocked by 4-PBA treatment either in TSC-1−/− or TSC-2−/− cells. Therefore, TSC-deficiency results in extreme sensitivity to glucose starvation induced apoptosis that originates from ER stress.

### Example 13

### Use of ER Stress Inducers to Promote Apoptosis of Tuberous Sclerosis Tumors

Since the tumors arising due to LOH in TSC-2+/− mice exhibit up-regulated UPR, we tested whether in vivo administration of thapsigargin will also selectively lead to apoptosis in the tumoral cells. To address this, we have used around ˜1 year old TSC-2+/− mice for thapsigargin (1 mg/kg) or vehicle treatment. After 7 days of thapsigargin administration we have analyzed the apoptosis with tunnel assay in the kidney adenomas. The tunnel assay positive cells showed a clear up-regulation in thapsigargin treated kidney adenomas. The lack of apoptotic cells either in thapsigargin treated TSC2+/− normal kidney tissue or vehicle treated adenomas indicates that the tumor cells are much more sensitive to develop apoptosis upon induction of ER stress. Increased tunnel positivity in liver hemangiomas after thapsigargin treatment was also observed, but lack of liver hemangiomas in vehicle treated group, limits the conclusions that can be drawn from the study.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A method of preventing, alleviating, and/or treating a hamartomatous disease comprising:
administering to a subject an endoplasmic reticulum (ER) stress modulating agent.

2. The method of claim 1, wherein the hamartomatous diseases is selected from the group consisting of tuberous sclerosis, pulmonary harnartoma, von Meyenburg complex, proteus syndrome, Birt-Dogg-Dube syndrome, multiple hamartoma syndrome, neurofibromatosis type 1, PeutzJeghers syndrome, Riley-Smith syndrome, and angiomyolipoma.

3. The method of claim 1, wherein the ER stress modulating agent is a chemical chaperone.

4. The method of claim 3, wherein the chemical chaperone is selected from the group consisting of glycerol, deuterated water (D₂O), dimethylsulfoxide (DMSO), trimethylamine N-oxide (TMAO), glycine betaine (betaine), glycerolphosphocholine (GPC), 4-phenyl butyrate or 4-phenyl butyric acid (PBA), methylamines, ursodeoxycholic acid (UDCA), and tauroursodeoxycholic acid (TUDCA).

5. The method of claim 1, wherein the ER stress modulating agent is an ER stress inducing agent.

6. The method of claim 5, wherein the ER stress inducing agent is selected from the group consisting of thapsigargin, tunicamycin, and azetidine-2 carboxylic acid (Azc).

7. The method of claim 3, wherein the agent is of the formula: 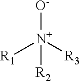
wherein
R₁, R₂, and R₃ are independently hydrogen, halogen, or lower C₁-C₆ alkyl; or
a pharmaceutically-acceptable salt thereof; or a mixture thereof.

8. The method of claim 7, wherein R1, R2, and R3 are independently lower C₁-C₆ alkyl.

9. The method of claim 3, wherein the agent is phenyl butyric acid (PBA).

10. The method of claim 9, wherein the agent is a derivative, salt, or isomer of PBA.

11. The method of claim 3, wherein the agent is of the formula: 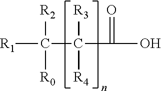
wherein n is 1 or 2;
R₀ is aryl, heteroaryl, or phenoxy, the aryl and phenoxy being unsubstituted or substituted with, independently, one or more halogen, hydroxyl, or lower alkyl;
R₁ and R₂ independently H, lower alkoxy, hydroxy, lower alkyl or halogen; and
R₃ and R₄ are independently H, lower alkyl, lower alkoxy or halogen; or a pharmaceutically-acceptable derivative or salt thereof.

12. The method of claim 11, wherein R₀ is phenyl, naphthyl, orphenoxy, the phenyl, naphthyl, and phenoxy being unsubstituted or substituted with, independently, one or more moieties of halogen, hydroxy or lower alkyl.

13. The method of claim 11, wherein
R₀ is phenyl, naphthyl, or phenoxy, the phenyl, naphthyl and phenoxy being unsubstituted or substituted with, independently, from 1 to 4 moieties of halogen, hydroxyl, or lower alkyl of from 1 to 4 carbon atoms;
R₁ and R₂ are, independently, H, hydroxy, lower alkoxy of from 1 to 2 carbon atoms, lower straight or branched chain alkyl of from 1 to 4 carbon atoms or halogen; and
R₃ and R₄ are, independently, lower alkoxy of from 1 to 2 carbon atoms, lower straight or branched chain alkyl of from 1 to 4 carbon atoms or halogen.

14. The method of claim 11, wherein n is 1.

15. The method of claim 11, wherein n is 2.

16. The method of claim 11, wherein R₀ is phenyl.

17. The method of claim 11, wherein R₀ is substituted phenyl.

18. The method of claim 11, wherein the substitution on the phenyl at R0 is from 1 to 4 halogen moieties.

19. The method of claim 11, wherein R3 and R4 are both —H.

20. The method of claim 3, wherein the agent is tauroursodeoxycholic acid (TUDCA).

21. The method of claim 3, wherein the agent is a derivative, salt, or isomer of TUDCA.

22. The method of claim 3, wherein the agent is of the formula: 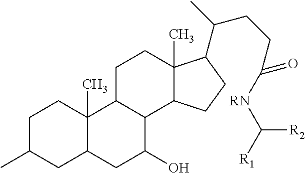
wherein R is —H or C₁-C₄ alkyl;
R₁ is —CH₂—SO₃R₃ and R₂ is —H; or R₁ is —COOH and R₂ is —CH₂—CH₂—CONH₂;
—CH₂—CONH₂, —CH₂—CH₂—SCH₃ or —CH₂—S—CH₂—COOH; and
R₃ is —H or the residue of a basic amino acid, or
a pharmaceutically acceptable salt or derivative thereof.

23. The method of claim 22, wherein R1 is —CH₂—SO₃H and R₂ is —H.

24. The method of claim 23, wherein R is —H.

25. The method of claim 1, further comprising diagnosing a subject as having a hamartomatous disease by detecting an increase in at least one marker of ER stress in a sample from the subject wherein the sample is suspected of containing cells under ER stress and comparing it to a control sample of cells not undergoing ER stress.

26. The method of claim 25, wherein the hamartomatous disease is selected from the group consisting of tuberous sclerosis, pulmonary hamartoma, von Meyenburg complex, proteus syndrome, Birt-Dogg-Dube syndrome, multiple hamartoma syndrome, neurofibromatosis type 1, Peutz Jeghers syndrome, Riley-Smith syndrome, and angiomyolipoma.

27. The method of claim 1, further comprising monitoring progression of the hamartomatous disease by monitoring a level of at least one marker indicative of ER stress.

28. The method of claim 25, wherein the markers indicative of ER stress is selected from the group consisting of spliced forms of XBP-1, phosphorylated PERK, phosphorylated eIF2α, phosphorylated IRE-1α, increased mRNA levels of GRP78/BIP, increased protein levels of GRP78/BIP, and increased JNK activation.

29. A method of screening for agents that modulate ER stress, the method comprising steps of:
providing an agent to be screened;
contacting the agent with at least one TSC-deficient cell; and
determining whether a level of at least one marker indicative of ER stress is changed as compared to at least one control cell.

30-38. (canceled)

39. A method of screening for agents that reduce ER stress, the method comprising steps of:
providing an agent to be screened;
contacting the agent with a TSC-deficient cell; and
determining whether at least one marker indicative of ER stress reduced.

40. (canceled)

41. A pharmaceutical composition comprising (1) an agent known to reduce ER stress, and (2) an agent selected from the group consisting of anti-neoplastic agents and anti-epileptic agents and a pharmaceutically acceptable carrier.

42. The pharmaceutical composition of claim 41, wherein the agent known to reduce ER stress is a chemical chaperone.

43. The pharmaceutical composition of claim 41, wherein the agent known to reduce ER stress is selected from the group consisting of dimethylsulfoxide (DMSO), glycine betaine (betaine), glycerolphosphocholine (OPC), methylamines, and trimethylamine N-oxide (TMAO).

44. The pharmaceutical composition of claim 41, wherein the agent known to reduce ER stress is TUDCA or a derivative thereof.

45. The pharmaceutical composition of claim 41, wherein the agent known to reduce ER stress is PBA or a derivative thereof.

46. The pharmaceutical composition of claim 41, comprising PBA and metformin.

47. A method of treating a tumor in a subject with tuberous sclerosis, the method comprising steps of:
administering to the subject a therapeutically effective amount of an agent that induces ER stress.

48. The method of claim 47, wherein the step of administering comprising administering the agent directly to the tumor.

49. The method of claim 48, wherein the agent is selected from the group consisting of thapsigargin, tunicamycin, and azetidine-2 carboxylic acid (Azc).

50. A packaged pharmaceutical comprising the pharmaceutical composition of claim 41 and instructions for treatment of a hamartomatous disease.

51. The package pharmaceutical of claim 50, wherein the hamartomatous disease is tuberous sclerosis.

52. A method for diagnosing a subject as having a hamartomatous disease comprising detecting an increase in at least one marker of ER stress in a sample from the subject wherein the sample is suspected of containing cells under ER stress and comparing it to a control sample of cells not undergoing ER stress.

53. A kit for screening for agents that modulate ER stress comprising at least one TSC-deficient cell line and instructions for use.
